(19) United States
(12) Patent Application Publication
(10) Pub. No.: US 2025/0147010 A1
(43) Pub. Date: May 8, 2025

(54) DETECTION AGENT, DETECTION SYSTEM AND METHOD THEREOF

(71) Applicant: NATIONAL CHENG KUNG UNIVERSITY, Tainan City (TW)

(72) Inventors: Guan-da SYU, Tainan City (TW); Tzong-shiann HO, Tainan City (TW); Tien-chun TSAI, Tainan City (TW)

(21) Appl. No.: 18/501,302

(22) Filed: Nov. 3, 2023

(51) Int. Cl. G01N 33/543; C07K 14/165; G01N 33/569; G01N 33/58

(52) U.S. Cl. CPC G01N 33/54326; C07K 14/165; G01N 33/56983; G01N 33/582; G01N 2333/165

(57) ABSTRACT

A detection agent, a detection system, and a method thereof are provided. The detection agent includes magnetic barcode beads. Each of the magnetic barcode beads has different two-dimensional edge and is conjugated with a corresponding protein. The protein includes a receptor binding domain of a spike protein or a nucleocapsid protein from a virus or a variant of the virus. The detection system includes the detection agent and a barcode bead fluorescence reader to read a fluorescence signal generated by each of the magnetic barcode beads. The method includes the steps of adding a serum of the subject to the detection agent followed by adding a fluorescently labeled anti-human immunoglobulin antibody and reading a fluorescence signal generated by each of the magnetic barcode beads and discriminating each of the magnetic barcode beads by the barcode bead fluorescence reader to quantify the anti-human immunoglobulin antibody.

### INCORPORATION BY REFERENCE OF SEQUENCE LISTING PROVIDED AS A XML FILE

The official copy of sequence listing is submitted concurrently with the specification as an XML file with a file name of TP230616-US-SEQUENCELIST.xml, a creation date of Oct. 25, 2023 and a size of 16,000 bytes. This sequence listing is part of the specification and is hereby incorporated in its entirely by reference herein.

### FIELD OF INVENTION

The present disclosure relates to the technical field of a detection agent, and particularly, to the detection agent comprising magnetic barcode beads conjugated with virus protein. The present disclosure also relates to the technical field of a detection system, and particularly, to the detection system comprising the detection agent. The present disclosure also relates to the technical field of a method, and particularly, to the method for profiling a humoral response in a subject by use of the detection system.

### BACKGROUND OF INVENTION

Coronavirus disease 2019” (COVID-19) pandemic caused by the severe acute respiratory syndrome coronavirus 2 (SARS-CoV-2) has been spread around the world since December 2019. Nowadays, COVID-19 vaccines such as mRNA-1273 vaccine developed by Moderna Inc and AZD1222 vaccine developed by the University of Oxford and AstraZeneca (AZ) company have been developed to protect people against COVID-19. Moreover, researchers continue studying and developing new COVID-19 vaccines. Therefore, COVID-19 infections and vaccinations are common worldwide, and it is important to distinguish the infected from vaccinations for many purposes, including tracking infections, epidemic investigations, vaccine research, public health tracking, etc.

Antibody detection may be used to evaluate the immune response of COVID-19 vaccinated subjects and COVID-19 patients and may be utilized in COVID-19 research. The conventional technology for detecting COVID-19 antibodies comprises enzyme-linked immunosorbent assay (ELISA) and lateral flow immunoassays (LFIA). However, although ELISA is preferred to be used in antibody detection for its high sensitivity and global adoption in clinical laboratories, ELISA may only detect a single strain of the virus at a time. In addition, for the multiplexed assay, the major drawback of the ELISA is that a large volume of a single specimen needs to be separated into different wells on the plate. Moreover, the LFIA for antibody detection has low sensitivity and specificity.

In order to analyze the humoral immunity against COVID-19 variants in a single test, a platform that can evaluate immune responses in a multiplexed manner is urgently needed. Protein microarray is a high-throughput technology that enables the detection of many proteins in parallel on a single chip. However, the drawbacks of protein microarray are the lack of automatic systems and the labor-intensive array alignments.

Therefore, development of an automatic, time-saving, low-cost, high-sensitivity, and high-throughput method to measure the antibody levels against multiple SARS-CoV-2 variants to evaluate the protection ability of COVID-19 vaccines against wild-type SARS-CoV-2 virus and several SARS-CoV-2 variants, classify the healthy subject and COVID-19 patients with mild/moderate, severe, and critical COVID-19, and distinguish the subjects vaccinated with COVID-19 vaccines from the subjects infected with COVID-19 are urgent problems to be solved in the art.

### SUMMARY OF INVENTION

To solve the problems mentioned above, one object of the present disclosure is to provide a detection agent for profiling a humoral response in a subject.

Another object of the present disclosure is to provide a detection kit for profiling a humoral response in a subject.

Still another object of the present disclosure is to provide a detection system for profiling a humoral response in a subject.

Still another object of the present disclosure is to provide a method for profiling a humoral response in a subject.

In order to achieve the objects mentioned above, the present disclosure provides a detection agent for profiling a humoral response in a subject. The detection agent comprises magnetic barcode beads. Each of the magnetic barcode beads has different two-dimensional edge and each of the magnetic barcode beads is conjugated with a corresponding protein.

The protein comprises a receptor binding domain (RBD) of a spike protein (S protein) or a nucleocapsid protein (N protein) from a virus or a variant of the virus.

The RBD of the S protein of the virus comprises an amino acid sequence of SEQ ID NO: 1.

The RBD of the S protein of the variant of the virus comprises, but is not limited to, an amino acid sequence of SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9.

In one embodiment, the virus comprises wild-type severe acute respiratory syndrome coronavirus 2 (SARS-CoV-2) virus, and the variant of the virus comprises, but is not limited to, SARS-CoV-2 B.1.1.7 variant, SARS-CoV-2 B.1.351 variant, SARS-CoV-2 P1 variant, SARS-CoV-2 B.1.617.2 variant, SARS-CoV-2 B.1.1.529 variant, SARS-CoV-2 BA.2.12.2 variant, SARS-CoV-2 BA.4 variant, or SARS-CoV-2 BA.5 variant.

In one embodiment, the N protein of the virus has an amino acid sequence of SEQ ID NO: 10.

In one embodiment, the N protein of the variant of the virus has an amino acid sequence of SEQ ID NO: 11.

The present disclosure also provides a detection kit for profiling a humoral response in a subject. The detection kit comprises the detection agent.

The present disclosure also provides a detection system for profiling a humoral response in a subject. The detection system comprises the detection agent and a barcode bead fluorescence reader. The barcode bead fluorescence reader reads a fluorescence signal generated by each of the magnetic barcode beads.

In one embodiment, the barcode bead fluorescence reader discriminates each of the magnetic barcode beads by a visible light.

In one embodiment, the visible light comprises a light-emitting diode (LED).

The present disclosure provides a method for profiling a humoral response in a subject, comprising the steps of:
providing a detection agent of claim 1;
adding a blocking reagent to the detection agent for reaction;
providing a serum of the subject, adding the serum to the detection agent for reaction, and then washing;
providing a fluorescently labeled anti-human immunoglobulin antibody, adding the fluorescently labeled anti-human immunoglobulin antibody to the detection agent, and reacting followed by washing; and
reading a fluorescence signal generated by each of the magnetic barcode beads and discriminating each of the magnetic barcode beads by a barcode bead fluorescence reader to quantify the anti-human immunoglobulin antibody; and
evaluating the humoral response based upon quantity of the anti-human immunoglobulin antibody.

In one embodiment, each of the magnetic barcode beads is discriminated by a visible light.

In one embodiment, the visible light comprises a LED.

In one embodiment, the detection agent is incubated with the serum for 50 to 70 minutes.

In one embodiment, the detection agent is incubated with the fluorescently labeled anti-human immunoglobulin antibody for 50 to 70 minutes.

In one embodiment, the method further comprises a step of evaluating protection ability of a COVID-19 vaccine against wild-type SARS-CoV-2 or SARS-CoV-2 variant the humoral response based upon the humoral response or quantity of the anti-human immunoglobulin antibody.

In one embodiment, the SARS-CoV-2 variant comprises SARS-CoV-2 B.1.1.7 variant, SARS-CoV-2 B.1.351 variant, SARS-CoV-2 P1 variant, SARS-CoV-2 B.1.617.2 variant, SARS-CoV-2 B.1.1.529 variant, SARS-CoV-2 BA.2.12.2 variant, SARS-CoV-2 BA.4 variant, and SARS-CoV-2 BA.5 variant.

In one embodiment, the method further comprises a step of classifying healthy subjects and COVID-19 patients with mild/moderate, severe, and critical COVID-19 based upon the humoral response or quantity of the anti-human immunoglobulin antibody.

In one embodiment, the method further comprises a step of distinguishing the subject vaccinated with a COVID-19 vaccine from the subject infected with COVID-19 based upon the humoral response or quantity of the anti-human immunoglobulin antibody.

In one embodiment, the COVID-19 vaccine comprises, but is not limited to, mRNA-1273 vaccine or AZD1222 vaccine.

In one embodiment, a fluorescence used for the fluorescently labeled anti-human immunoglobulin antibody comprises cyanine dye Cy3 or cyanine dye Cy5.

The present application provides an automatic, time-saving, low-cost, high-sensitivity, and high-throughput detection agent, detection system, and method to measure the antibody levels against wild-type SARS-CoV-2 virus and SARS-CoV-2 variants using magnetic barcode beads conjugated with RBD of S protein or N protein from wild-type SARS-CoV-2 virus and several SARS-CoV-2 variants. Based on the two-dimensional edges of the magnetic barcode beads, the barcode beads fluorescence reader may accurately distinguish different magnetic barcode beads as well as quantify the fluorescence intensity on every individual magnetic barcode bead.

Moreover, the detection system of the present application is utilizing the visible light such as LED for discriminating the magnetic barcode beads with two-dimensional edges rather than using fluorescence-coded beads. Therefore, the barcode bead fluorescence system does not require a complex fluorescence system. In addition, the barcode bead fluorescence system may multiplex up to 1000 magnetic barcode beads.

Furthermore, the detection system of the present application with advantages in more multiplex capability, high throughput screening, convenience, and cost-effectiveness may be used to profile the humoral responses of subjects for evaluating the protection ability of COVID-19 vaccines against wild-type SARS-CoV-2 or SARS-CoV-2 variants, classifying the healthy subject and COVID-19 patients with mild/moderate, severe, and critical COVID-19, and distinguishing the subjects vaccinated with COVID-19 vaccines from the subjects infected with COVID-19 quickly and accurately by profiling humoral responses of the subjects.

### BRIEF DESCRIPTION OF DRAWINGS

FIG. 1A shows a top view of a scanning electron microscope (SEM) image of a magnetic barcode bead under the focused ion beam (FIB) etching.

FIG. 1B shows a cross-section view of the SEM image and an energy dispersive spectrometer (EDS) analysis of the magnetic barcode bead under the FIB etching. Point 1 (glass) and point 2 (magnetic layer) are used for the EDS analysis.

FIG. 2 shows the representative images of each magnetic barcode bead in one well of 96-well plate. Twelve mixed magnetic barcode beads may be distinguished in a single well via a barcode bead fluorescence (BBF) reader. Each magnetic barcode bead is enlarged. The imaging process takes about 30 sec for one well for both white light and fluorescence channels.

FIG. 3A shows a process of a BBF assay of the present application.

FIG. 3B shows the results of reproducibility of two independent anti-His fluorescence staining.

FIG. 3C shows that serial dilutions of anti-spike antibodies are used to show a detection curve of the magnetic barcode bead conjugated with receptor binding domain (RBD) from wild-type SARS-CoV-2 virus. Relative fluorescence intensity (RFI) indicated the fluorescence intensity relative to the anti-His signal.

FIG. 3D shows the results of comparison between anti-RBD RFI from the BBF assay of the present application and anti-RBD BAU/mL provided by the WHO reference panel. The equation of the regression is Y=18.57*X+122.9.

FIG. 4A to FIG. 41 shows the results of antibodies of the serum collected from unvaccinated subjects (UV), subjects vaccinated with two doses of AZD1222 (2×AZ) vaccine, and subjects vaccinated with two doses of mRNA-1273 (2×M2) vaccine against RBD from wild-type SARS-CoV-2 virus and eight SARS-CoV-2 variants. Data are analyzed by one-way ANOVA followed by Dunn's posthoc tests, ****p<0.0001, ***p<0.001, and **p<0.01 as indicated comparisons.

FIG. 5A to FIG. 5I show the results of antibodies of the serum collected from healthy subject (H) and COVID-19 patients with mild/moderate (M), severe (S), and critical (C) cases against RBD from wild-type SARS-CoV-2 virus and eight SARS-CoV-2 variants. Data are analyzed by one-way ANOVA followed by Dunn's posthoc tests, ****p<0.0001, ***p<0.001, **p<0.01, and *p<0.05 as indicated comparisons.

FIG. 6A to FIG. 6C show the results of antibodies of the serum collected from COVID-19 patients with mild/moderate (M), severe (S), and critical (C) cases, respectively against RBD from wild-type SARS-CoV-2 virus and eight SARS-CoV-2 variants. Data are analyzed by one-way ANOVA followed by Dunn's posthoc tests, ****p<0.0001, ***p<0.001, **p<0.01, and *p<0.05 as indicated comparisons.

FIG. 7A and FIG. 7B show the results of antibodies of the serum collected from unvaccinated subjects (UV), subjects vaccinated with two doses of AZD1222 (2×AZ) vaccine, and subjects vaccinated with two doses of mRNA-1273 (2×M2) vaccine against nucleocapsid (N) protein from wild-type SARS-CoV-2 virus and SARS-CoV-2 Omicron B.1.1.529 variant, respectively. Data are analyzed by one-way ANOVA followed by Dunn's posthoc tests, ****p<0.0001, ***p<0.001, and **p<0.01 as indicated comparisons.

FIG. 7C and FIG. 7D show the results of antibodies of the serum collected from healthy subject (H) and COVID-19 patients with mild/moderate (M), severe (S), and critical (C) cases against N protein from wild-type SARS-CoV-2 virus and SARS-CoV-2 Omicron B.1.1.529 variant, respectively. Data are analyzed by one-way ANOVA followed by Dunn's posthoc tests, ****p<0.0001, ***p<0.001, and **p<0.01 as indicated comparisons.

### DETAILED DESCRIPTION OF PREFERRED EMBODIMENTS

The following provides specific embodiments to illustrate the implementation of the present disclosure. A person having ordinary skill in the art can understand other advantages and effects of the present disclosure from the contents disclosed in the present specification. However, the exemplary embodiments disclosed in the present disclosure are only for illustrative purposes and should not be regarded as limiting the scope of the present disclosure. In other words, the present disclosure can also be implemented or applied by other different specific embodiments, and various details in the present specification can also be modified and changes based on different viewpoints and applications without departing from the concept of the present disclosure.

Unless otherwise indicated herein, the singular forms “one” and “the” used in the specification and the appended claims of the present disclosure include the plural. Unless otherwise indicated herein, the term “or” used in the specification and the appended claims of the present disclosure includes the meaning of “and/or”.

### Preparation Example 1: Preparation of Magnetic Barcode Beads Conjugated with RBD of S Protein or N Protein from Wild-Type SARS-CoV-2 Virus and Several SARS-CoV-2 Variants

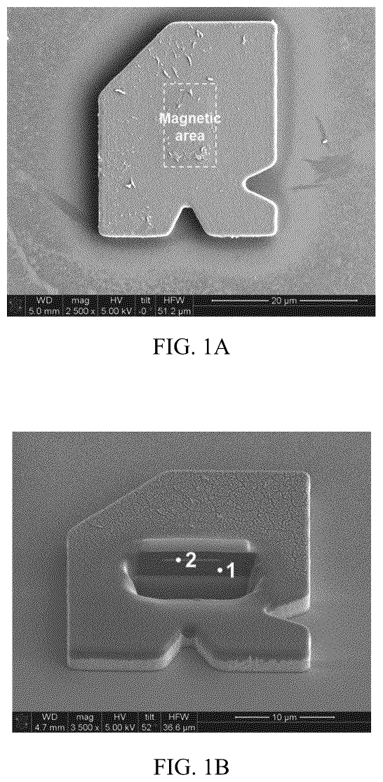

Referring to FIG. 1A and FIG. 1B, a magnetic barcode bead is used for barcode bead fluorescence (BBF) assay. The results of energy dispersive spectrometer (EDS) analysis are shown in Table 1. The encapsulated magnetic layer of nickel oxide is the same pattern, which is applied for convenient specimen pretreatment.

TABLE 1
The results of EDS analysis of the magnetic barcode bead

| Element (At %) | Si | O | Ni |
|---|---|---|---|
| Point 1 | 43.53 | 56.47 | 0 |
| Point 2 | 18.17 | 41.75 | 40.08 |

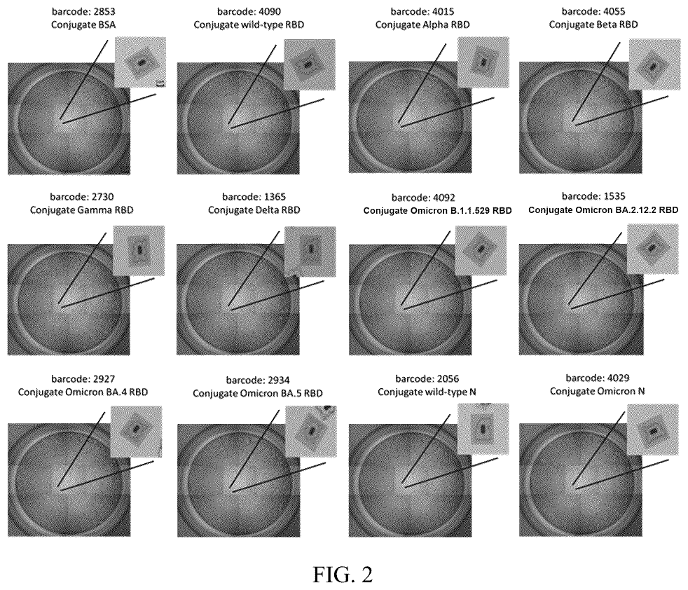

The sensing mechanism of the BBF multiplexed assay is mainly based on the outer edges of the magnetic barcode beads rather than the inner magnetic layer, which are patterned by the photolithography. Referring to FIG. 2, each magnetic barcode bead (WinMEMS Technologies, Taiwan) is different in the two-dimensional edges and can be defined as digital numbers by a barcode beads fluorescence reader. Based on the two-dimensional edges, the barcode beads fluorescence reader can accurately distinguish different magnetic barcode beads as well as quantify the fluorescence intensity on every individual magnetic barcode bead.

For example, magnetic barcode beads of barcode 2853, 4090, 4015, 4055, 2730, 1365, 4092, 1535, 2927, 2934, 2056, and 4029 are used conjugated with BSA, RBD from wild-type SARS-CoV-2 virus, RBD from SARS-CoV-2 alpha variant (B.1.1.7), RBD from SARS-CoV-2 beta variant (B.1.351), RBD from SARS-CoV-2 gamma variant (P1), RBD from SARS-CoV-2 delta variant (B.1.617.2), RBD from SARS-CoV-2 omicron variant (B.1.1.529), RBD from SARS-CoV-2 omicron variant (BA.2.12.2), RBD from SARS-CoV-2 omicron variant (BA.4), RBD from SARS-CoV-2 omicron variant (BA.5), N protein from wild-type SARS-CoV-2 virus, and N protein from SARS-CoV-2 omicron variant (B.1.1.529), respectively.

The magnetic barcode beads with different barcodes are modified with NHS/EDC reagents (Thermo Fisher, No. 24510/22980) for 30 min to conjugate the proteins with amine group and washed 3 times with a magnetic rack (Thermo Fisher, No. 12321D). Each protein shown in Table 2 is incubated with magnetic barcode beads with a unique barcode overnight at 4° C. (2.5 μg for 10\hat{ }5 beads) and washed 3 times with the magnetic rack.

Bovine serum albumin (BSA) shown in Table 2 is purchased from Sigma-Aldrich (USA) and eleven proteins shown in Table 2 including receptor binding domain (hereinafter referred to as “RBD”) of spike protein (hereinafter referred to as “S protein”) from wild-type SARS-CoV-2 virus and several SARS-CoV-2 variants with histidine tag (His-tag), and nucleocapsid proteins (hereinafter referred to as “N protein”) from wild-type SARS-CoV-2 virus and SARS-CoV-2 variant with His-tag are purchased from Sino Biological Inc. (Mainland China). Each protein is immobilized on the magnetic barcode beads and the amine group of 9 spike RBDs, 2 nucleocapsids, and BSA are conjugated onto corresponding magnetic barcode beads. The magnetic barcode beads conjugated with RBD of S protein or N protein from wild-type SARS-CoV-2 virus and several SARS-CoV-2 variants (also referred to as “conjugated magnetic barcode beads”) are then resuspended in the 1.5 ml storage buffer (comprising 1% BSA, 0.0100 sodium azide, and 5000 glycerol in PBST) and stored at 4° C. or −80° C. for subsequent use.

TABLE 2
Animo acid sequence of RBD of S protein from wild-type
SARS-CoV-2 virus and several SARS-CoV-2 variants
with His-tag and N protein from wild-type SARS-CoV-
2 virus and SARS-CoV-2 variant with His-tag.

| | | | Amino Acid |
|---|---|---|---|
| | Virus | | Sequence |
| BSA | — | — | — |
| RBD | Wild-type | SARS-CoV-2 virus | SEQ ID NO: 1 |
| | SARS- | B.1.1.7 (i.e., α variant) | SEQ ID NO: 2 |
| | CoV-2 | B.1.351 (i.e., β variant) | SEQ ID NO: 3 |
| | variant | P1 (i.e., γ variant) | SEQ ID NO: 4 |
| | | B.1.617.2 (i.e., δ variant) | SEQ ID NO: 5 |
| | | B.1.1.529 (i.e., Omicron | SEQ ID NO: 6 |
| | | variant) | |
| | | BA.2.12.2 (i.e., Omicron | SEQ ID NO: 7 |
| | | variant) | |
| | | BA.4 (i.e., Omicron variant) | SEQ ID NO: 8 |
| | | BA.5 (i.e., Omicron variant) | SEQ ID NO: 9 |
| N | Wild-type | SARS-CoV-2 virus | SEQ ID NO: 10 |
| protein | SARS- | B.1.1.529 (i.e., Omicron | SEQ ID NO: 11 |
| | CoV-2 | variant) | |
| | variant | | |

### Example 1: Examination the Quality of Magnetic Barcode Beads Conjugated with RBD of S Protein or N Protein from Wild-Type SARS-CoV-2 Virus and Several SARS-CoV-2 Variants for Barcode Bead Fluorescence (BBF) Assay

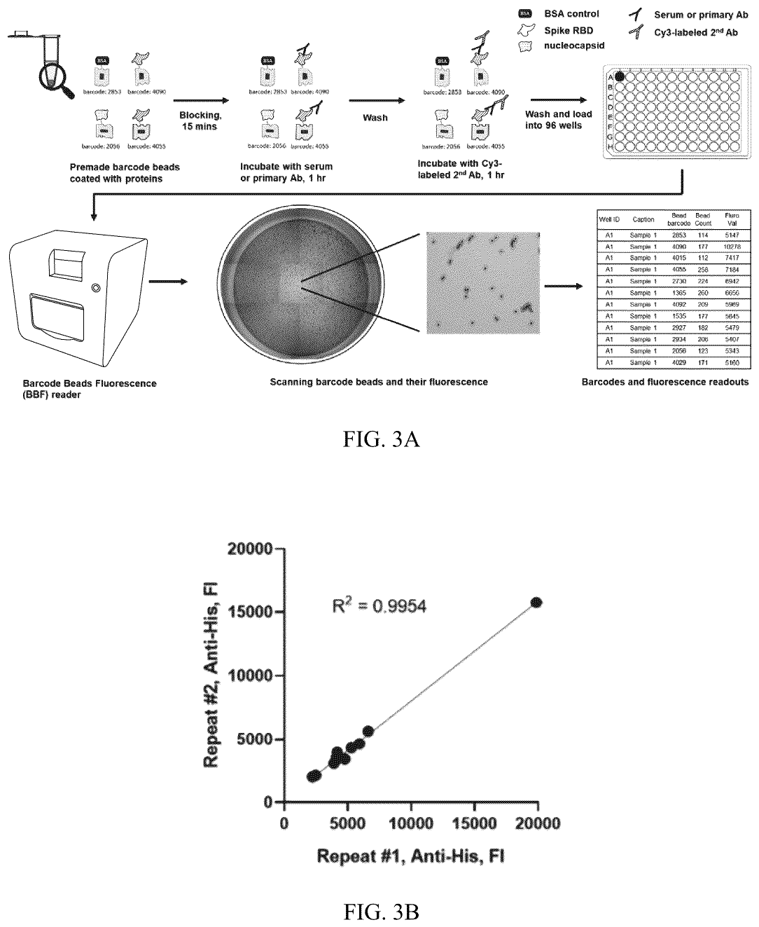

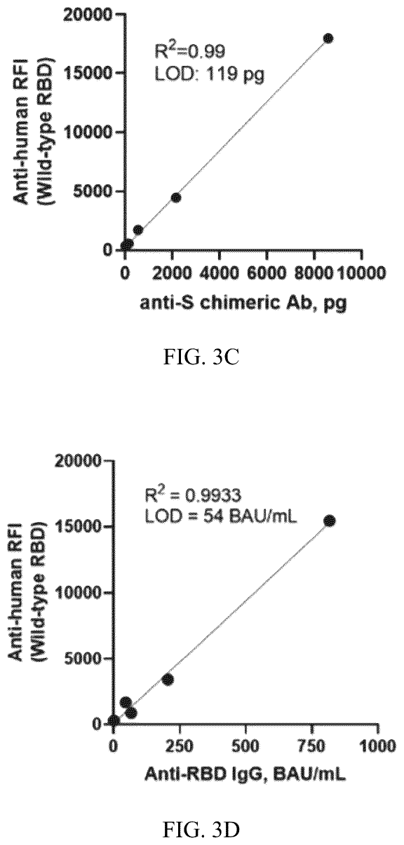

Referring to FIG. 3A, the conjugated magnetic barcode beads are blocked with superblock (Thermo Fisher, No. WL337222) for 15 minutes, incubated with 50 μL anti-His tag mouse monoclonal antibody for 1 hour, washed with PBST buffer, incubated again with 50 μL Cy3-labeled anti-mouse IgG antibody (0.015 mg/ml, Jackson Laboratory, No. 115-165-003) for 1 hour, and washed with PBST buffer. The conjugated magnetic barcode beads are transferred to a clear 96-well plate for reading. The barcode beads fluorescence reader (BBF reader, VISIONATICS, Taiwan) is used to acquire the fluorescence intensities and the magnetic barcode beads with two-dimensional edges may be discriminated by a light-emitting diode (LED). For the relative fluorescence intensity (RFI), fluorescence signals are subtracted with BSA and then normalized to the anti-His signals. The limit of detection (LOD) is defined as a 3-fold standard error of Y-intercept/best-fit value slope.

While 12 different conjugated magnetic barcode beads are mixed in one well, the BBF reader can decrypt each conjugated magnetic barcode bead based on different two-dimensional edges. The conjugated magnetic barcode bead counts ranged from 50 to 200 and shows good reproducibility in two experiments (data not shown).

To verify the immobilization of the protein, the conjugated magnetic barcode beads are stained with anti-His and Cy3-labeled anti-mouse antibodies. Referring FIG. 3B, the results show that the reproducibility of anti-His signals is R²=0.9954. Moreover, a serial dilution of the anti-spike chimeric monoclonal antibody (Sino Biological, No. 40150-D004) against RBD from wild-type SARS-CoV-2 virus is used to calculate the detection limit. Referring FIG. 3C, the results show that the calculated LOD is 119 μg with R²=0.99. Therefore, the BBF platform is a high throughput and a reproducing method for profiling molecular interactions.

To validate the BBF assays, the WHO reference panel which provides serums (NIBSC code: 20/268) with binding antibody units (BAU) is used to set up the serum assay again RBD from wild-type SARS-CoV-2. Five standard serums ranging from high to low reactivities to the SARS-CoV-2 (anti-RBD levels are 817, 205, 66, 45, and 0 BAU/mL) are provided. After blocking, the magnetic barcode beads conjugated with wild-type RBD are incubated with 50 μL of 250-fold diluted serum for 1 hour. After washing with PBST buffer, the magnetic barcode beads conjugated with wild-type RBD are incubated with 50 μL Cy3-labeled anti-human antibody for 1 hour, followed by being washed, loaded into 96 wells, and quantified by the BBF reader.

Referring to FIG. 3D, the results show that the RFI from the serum assay significantly correlated with BAU/ml provided by the supplier with R²=0.9933 and LOD=54 BAU/ml.

### Example 2: Examination the Antibody Responses of Subjects after COVID-19 Vaccinations by BBF Assay

To examine the immune response of subjects vaccinated against COVID-19, the serum is collected from unvaccinated subjects (UV, N=16), subjects vaccinated with two doses of AZD1222 vaccine (2×AZ, N=20), and subjects vaccinated with two doses of mRNA-1273 vaccine (2×M2, N=14). Subjects who had a diagnosis of COVID-19 history are excluded from the vaccine study. As shown in Table 3, the average days after vaccination are 59±24 in 2×AZ and 57±28 in 2×M.

TABLE 3
Basic characteristics of unvaccinated and vaccinated subjects.

| | | Two doses | Two doses |
|---|---|---|---|
| | Unvaccinated | AZD1222 | mRNA- |
| Classifications | (UV) | (2xAZ) | 1273 (2xM) |
| Total number (N) | 16 | 20 | 14 |
| Age (years, SD) | 63 (11) | 37 (7) | 36 (5) |
| Gender (male, %) | 7 (43%) | 5 (25%) | 2 (14%) |
| The days after vaccination | — | 59 (24) | 57 (28) |
| (days, SD) | | | |

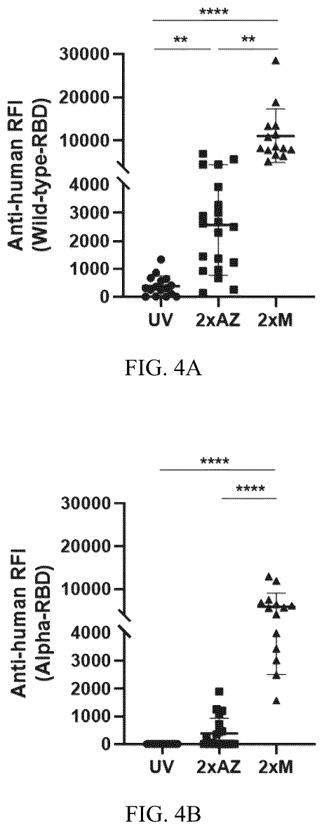

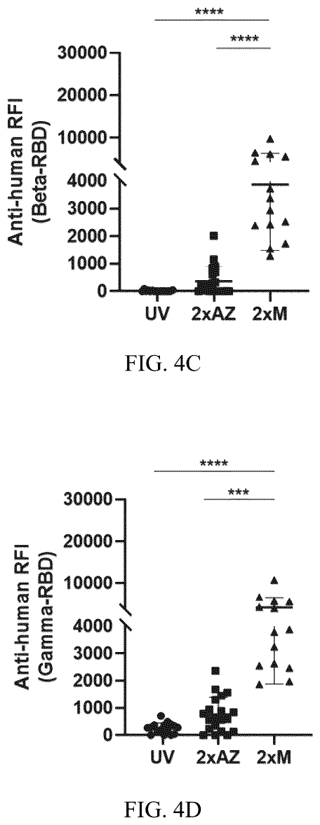

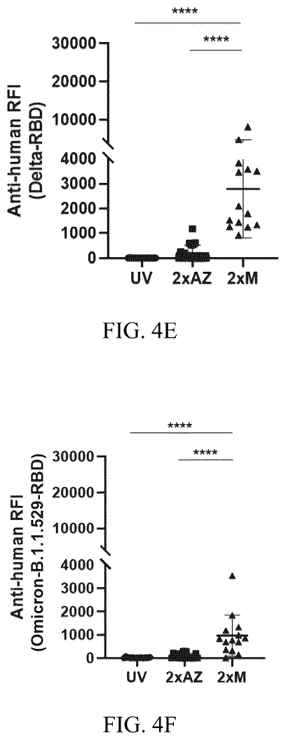

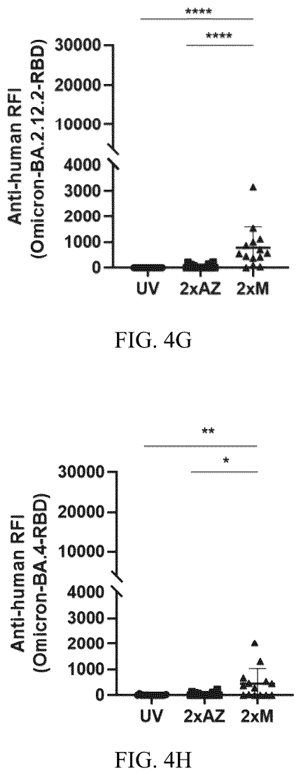

The serum antibodies against RBDs from wild-type SARS-CoV-2 and SARS-CoV-2 variants are profiled. The antibody levels in the UV group, 2×AZ group, and 2×M group are quantified by anti-human fluorescence. Referring to FIG. 4A, the results show that the increased antibody is observed in both 2×AZ group and 2×M group against wild-type RBD. Referring to FIG. 4B to FIG. 41, the results show that for the SARS-CoV-2 variant RBDs, the elevation of the 2×AZ group is abolished while 2×M group remained higher compared to the UV group. The binding antibodies are decreased in SARS-CoV-2 variants and almost undetectable in SARS-CoV-2 omicron variant. Thus, stronger antibody responses and unique patterns characterize the 2×M compared to the 2×AZ and UV. Therefore, the BBF assay may be used to evaluate the protection ability of COVID-19 vaccines against wild-type SARS-CoV-2 or SARS-CoV-2 variants.

### Example 3: Examination the Antibody Responses in COVID-19 Patients with Different Severities by BBF Assay

To show the potential link between humoral responses and COVID-19 outcomes, the antibody responses in COVID-19 patients with different severities are performed by use of BBF assay. Sera from healthy subject (H group, N=16) and COVID-19 patients with mild/moderate (M group, N=16), severe (S group, N=16), and critical (C group, N=16) cases are collected from NHRI Biobank without receiving any COVID-19 vaccines. The days between symptom onset and draw are 36±11 in M group, 26±13 in S group, and 6±1 in C group. The basic characteristics of healthy subject and COVID-19 patients with mild/moderate, severe, and critical COVID-19 are shown in Table 4. Since the NURI biobank randomly collected the COVID-19 patients, the basic characteristics of the COVID-19 patients are compared for the risk of critical COVID-19 (50% are deceased in the C group). The results show age and diabetes are significantly enriched in the critical group.

TABLE 4
Basic characteristics of healthy and COVID-19 patients.

| | | | | | p-value | | |
|---|---|---|---|---|---|---|---|
| | | | | | M group | S group | M group |
| | | | | | vs C | vs C | vs S |
| Classifications | H group | M group | S group | C group | group | group | group |
| Total number (N) | 16 | 16 | 16 | 16 | — | — | — |
| Age (years, SD) | 63 (11) | 38 (13) | 54 (17) | 76 (8) | 0.0001 | 0.0058 | 0.1079 |
| Gender (male, %) | 7 (43%) | 8 (50%) | 5 (31%) | 8 (50%) | 1.0000 | 0.2802 | 0.2802 |
| Deceased (N, %) | 0 (0%) | 0 (0%) | 0 (0%) | 8 (50%) | 0.0011 | 0.0011 | 1.0000 |
| Diabetes (N, %) | 0 (0%) | 1 (6%) | 1 (6%) | 8 (50%) | 0.0059 | 0.0059 | 1.0000 |
| Hypertension (N, %) | 0 (0%) | 1 (6%) | 5 (31%) | 4 (25%) | 0.1441 | 0.6942 | 0.0700 |
| Cardiovascular | 0 (0%) | 0 (0%) | 2 (12%) | 2 (12%) | 0.1441 | 1.0000 | 0.1441 |
| disease (N, %) | | | | | | | |

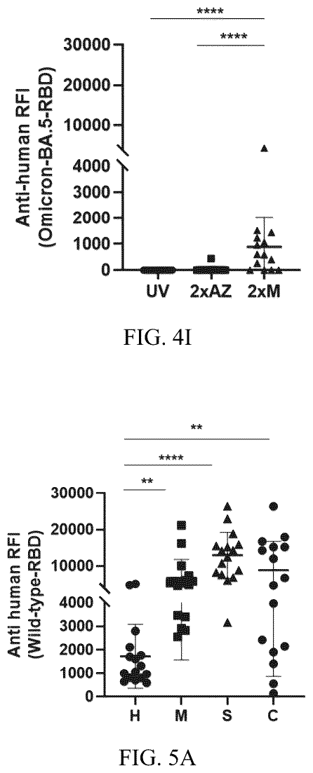

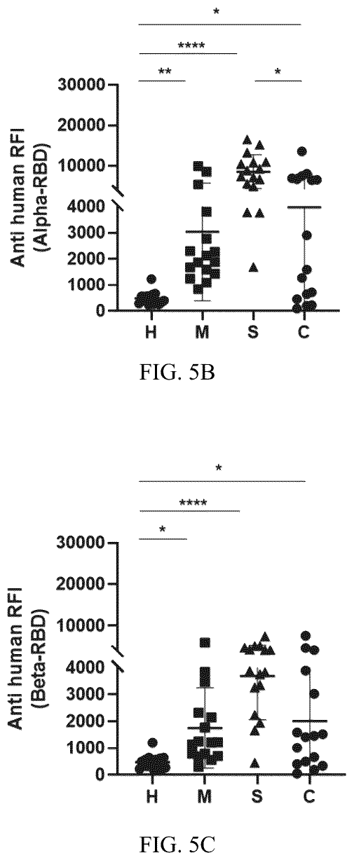

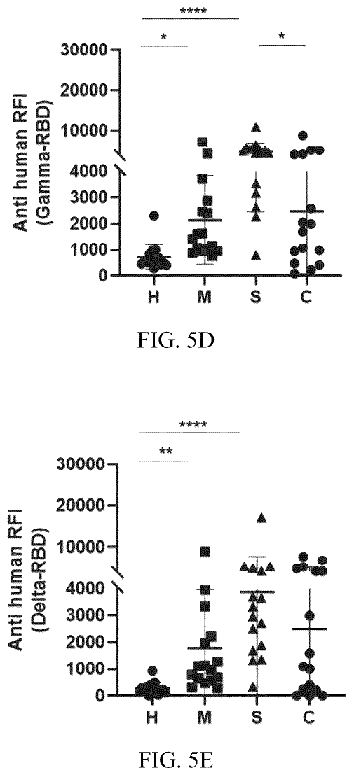

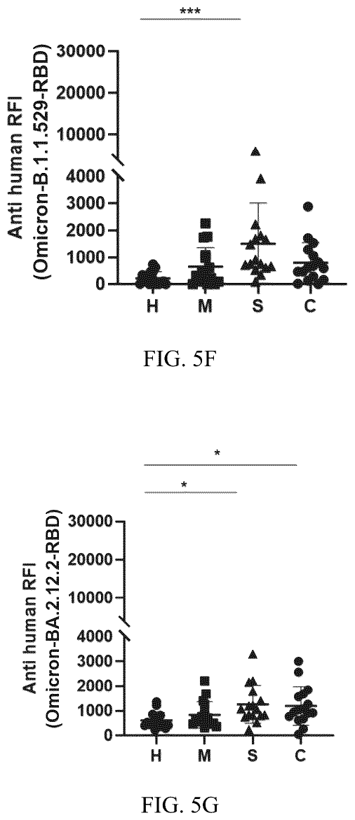

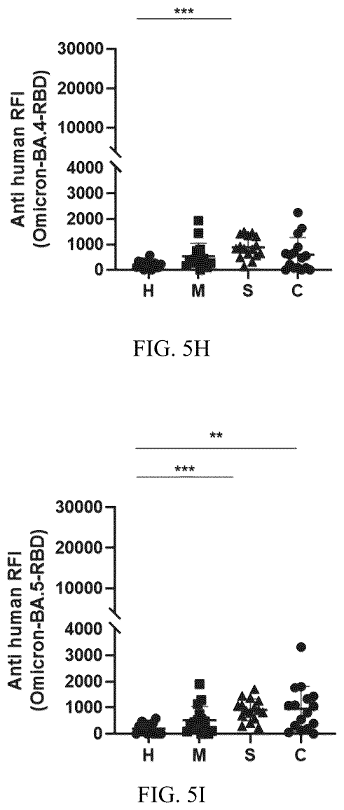

The antibody levels in the serum of COVID-19 patients with different severities are quantified by anti-human fluorescence. Referring to FIG. 5A to FIG. 5C and FIG. 5E, the results show that the antibody level against the magnetic barcode beads conjugated with RBDs from wild-type SARS-CoV-2 and SARS-CoV-2 variants, especially from wild-type SARS-CoV-2 and SARS-CoV-2 alpha, beta, and delta variants, is higher in COVID-19 patients. Referring to FIG. 5A to FIG. 5I, among the COVID-19 patients with different severities, the S group showed a higher trend of antibody levels. However, there are no differences between the M group and C group.

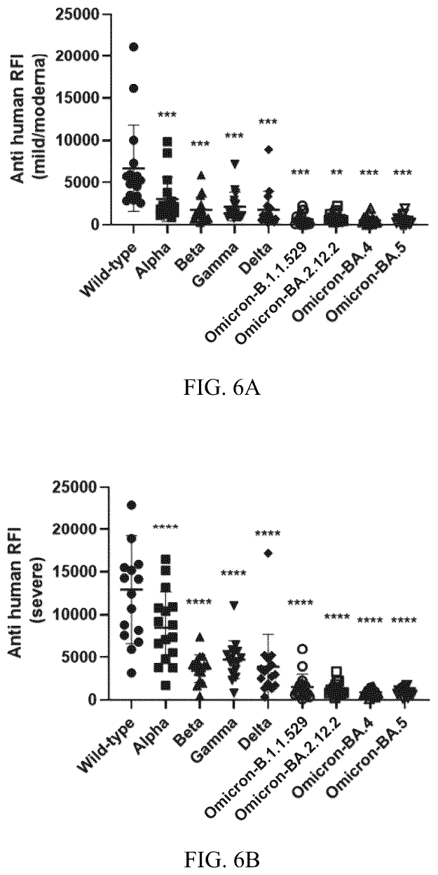

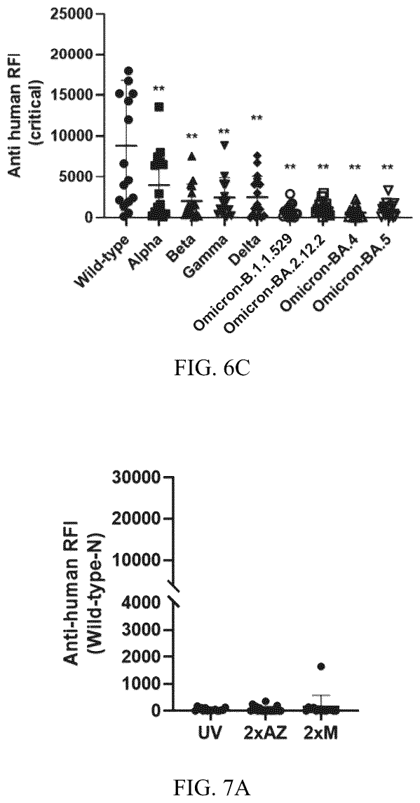

Moreover, referring to FIG. 6A to 6C, the results show that the binding antibodies are reduced in SARS-CoV-2 variants and almost undetectable in SARS-CoV-2 omicron variants. The antibody level in the S group showed a distinct pattern compared to the M group, which might be useful for classifications. Therefore, the BBF assay may be used to classify the healthy subject and COVID-19 patients with mild/moderate, severe, and critical COVID-19.

### Example 4: Distinguish the Subjects Vaccinated with COVID-19 Vaccines from the Subjects Infected with COVID-19 by BBF Assay

The N protein of SARS-CoV-2 virus is crucial for virus genome packaging. Due to the COVID-19 vaccine targeting the S protein, the antibodies in the serum of subjects against the N protein may only be detected after natural infections.

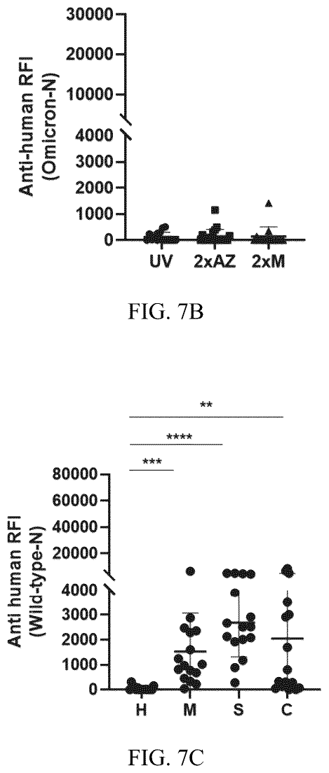

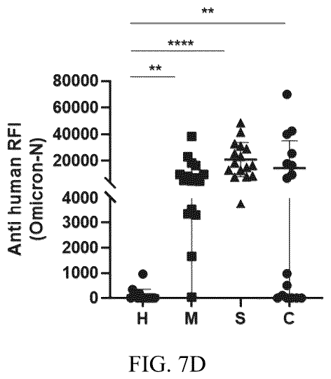

The antibody levels in the serum of unvaccinated subjects, subjects vaccinated with COVID-19 vaccines, healthy subjects, and subjects infected with COVID-19 with different severities are quantified by anti-human fluorescence. Referring to FIGS. 7A and 7B, the results demonstrate no elevation of antibodies against the magnetic barcode beads conjugated with N protein from wild-type SARS-CoV-2 and SARS-CoV-2 omicron variant compared to the unvaccinated subjects. Conversely, referring to FIGS. 7C and 7D, the results show that the subjects infected with COVID-19 with different severities show elevated antibodies against the magnetic barcode beads conjugated with N protein from wild-type SARS-CoV-2 and SARS-CoV-2 omicron variant compared to the healthy subjects. Therefore, the BBF assay may be used to distinguish the subjects vaccinated with COVID-19 vaccines from the subjects infected with COVID-19.

From the above, the present application provides an automatic, time-saving, low-cost, high-sensitivity, and high-throughput detection agent, detection system, and method to measure the antibody levels against wild-type SARS-CoV-2 virus and SARS-CoV-2 variants using magnetic barcode beads conjugated with RBD of S protein or N protein from wild-type SARS-CoV-2 virus and several SARS-CoV-2 variants. Based on the two-dimensional edges of the magnetic barcode beads, the barcode beads fluorescence reader may accurately distinguish different magnetic barcode beads as well as quantify the fluorescence intensity on every individual magnetic barcode bead.

Moreover, the barcode bead fluorescence system of the present application is utilizing the visible light such as LED for discriminating the magnetic barcode beads with two-dimensional edges rather than using fluorescence-coded beads. Therefore, the barcode bead fluorescence system does not require a complex fluorescence system. In addition, the barcode bead fluorescence system may multiplex up to 1000 magnetic barcode beads.

Furthermore, the barcode bead fluorescence system of the present application with advantages in more multiplex capability, high throughput screening, convenience, and cost-effectiveness may be used to profile the humoral responses of subjects for evaluating the protection ability of COVID-19 vaccines against wild-type SARS-CoV-2 or SARS-CoV-2 variants, classifying the healthy subject and COVID-19 patients with mild/moderate, severe, and critical COVID-19, and distinguishing the subjects vaccinated with COVID-19 vaccines from the subjects infected with COVID-19 quickly and accurately by profiling humoral responses of the subjects.

Although the present disclosure has been disclosed in preferred embodiments, it is not intended to limit the present disclosure. A person having ordinary skill in the art can make various changes and modifications without departing from the concept and scope of the present disclosure. Therefore, the claimed scope of the present disclosure shall be based on the scope defined by the attached claims of the patent disclosure.

What is claimed is:

1. A detection agent for profiling a humoral response in a subject, comprising magnetic barcode beads, wherein each of the magnetic barcode beads has different two-dimensional edge and each of the magnetic barcode beads is conjugated with a corresponding protein;
wherein the protein comprises a receptor binding domain of a spike protein or a nucleocapsid protein from a virus or a variant of the virus;
wherein the receptor binding domain of the spike protein of the virus comprises an amino acid sequence of SEQ ID NO: 1;
the receptor binding domain of the spike protein of the variant of the virus comprises an amino acid sequence of SEQ ID NO: 2, SEQ ID NO: 3, SEQ ID NO: 4, SEQ ID NO: 5, SEQ ID NO: 6, SEQ ID NO: 7, SEQ ID NO: 8, or SEQ ID NO: 9.

2. The detection agent according to claim 1, wherein the virus comprises wild-type severe acute respiratory syndrome coronavirus 2 (SARS-CoV-2) virus, and the variant of the virus comprises SARS-CoV-2 B.1.1.7 variant, SARS-CoV-2 B.1.351 variant, SARS-CoV-2 P1 variant, SARS-CoV-2 B.1.617.2 variant, SARS-CoV-2 B.1.1.529 variant, SARS-CoV-2 BA.2.12.2 variant, SARS-CoV-2 BA.4 variant, or SARS-CoV-2 BA.5 variant.

3. The detection agent according to claim 1, wherein the nucleocapsid protein of the virus has an amino acid sequence of SEQ ID NO: 10.

4. The detection agent according to claim 1, wherein the nucleocapsid protein of the variant of the virus has an amino acid sequence of SEQ ID NO: 11.

5. A detection system for profiling a humoral response in a subject, comprising a detection agent of claim 1 and a barcode bead fluorescence reader, wherein the barcode bead fluorescence reader reads a fluorescence signal generated by each of the magnetic barcode beads.

6. The detection system according to claim 5, wherein the barcode bead fluorescence reader discriminates each of the magnetic barcode beads by a visible light.

7. The detection system according to claim 6, wherein the visible light comprises a light-emitting diode.

8. A method for profiling a humoral response in a subject, comprising the steps of:
providing a detection agent of claim 1;
adding a blocking reagent to the detection agent for reaction;
providing a serum of the subject, adding the serum to the detection agent for reaction, and then washing;
providing a fluorescently labeled anti-human immunoglobulin antibody, adding the fluorescently labeled anti-human immunoglobulin antibody to the detection agent, and reacting followed by washing; and
reading a fluorescence signal generated by each of the magnetic barcode beads and discriminating each of the magnetic barcode beads by a barcode bead fluorescence reader to quantify the anti-human immunoglobulin antibody.

9. The method according to claim 8, wherein each of the magnetic barcode beads is discriminated by a visible light.

10. The method according to claim 9, wherein the visible light comprises a light-emitting diode.

11. The method according to claim 8, wherein the serum incubates with the detection agent for 50 to 70 minutes.

12. The method according to claim 8, wherein the fluorescently labeled anti-human immunoglobulin antibody incubates with the detection agent for 50 to 70 minutes.

13. The method according to claim 8, wherein the humoral response is used to evaluate protection ability of a COVID-19 vaccine against wild-type SARS-CoV-2 or SARS-CoV-2 variant.

14. The method according to claim 13, wherein the SARS-CoV-2 variant comprises SARS-CoV-2 B.1.1.7 variant, SARS-CoV-2 B.1.351 variant, SARS-CoV-2 P1 variant, SARS-CoV-2 B.1.617.2 variant, SARS-CoV-2 B.1.1.529 variant, SARS-CoV-2 BA.2.12.2 variant, SARS-CoV-2 BA.4 variant, and SARS-CoV-2 BA.5 variant.

15. The method according to claim 8, wherein the humoral response is used to classify healthy subjects and COVID-19 patients with mild/moderate, severe, and critical COVID-19.

16. The method according to claim 8, wherein the humoral response is used to distinguish the subject vaccinated with a COVID-19 vaccine from the subject infected with COVID-19.

17. The method according to claim 16, wherein the COVID-19 vaccine comprises mRNA-1273 vaccine or AZD1222 vaccine.

18. The method according to claim 8, wherein a fluorescence used for the fluorescently labeled anti-human immunoglobulin antibody comprises cyanine dye Cy3 or cyanine dye Cy5.


SEQUENCE LISTING (11 sequences)

SEQ ID NO: 1 - protein, 222 aa, Betacoronavirus Severe acute respiratory syndrome-related coronavirus
  source 1..222: note = WT-receptor binding domain
VQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 2 - protein, 222 aa, Betacoronavirus Severe acute respiratory syndrome-related coronavirus
  source 1..222: note = B.1.1.7-Receptor binding domain
VQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNF

SEQ ID NO: 3 - protein, 222 aa, Betacoronavirus Severe acute respiratory syndrome-related coronavirus
  source 1..222: note = B.1.351-receptor binding domain
VQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNF

SEQ ID NO: 4 - protein, 222 aa, Betacoronavirus Severe acute respiratory syndrome-related coronavirus; identical to SEQ ID NO: 3
  source 1..222: note = P1-receptor binding domain- P1 variant

SEQ ID NO: 5 - protein, 222 aa, Betacoronavirus Severe acute respiratory syndrome-related coronavirus
  source 1..222: note = B.1.617.2-receptor binding domain
VQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNF

SEQ ID NO: 6 - protein, 222 aa, Betacoronavirus Severe acute respiratory syndrome-related coronavirus
  source 1..222: note = B.1.1.529-receptor binding domain
VQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNF

SEQ ID NO: 7 - protein, 222 aa, Betacoronavirus Severe acute respiratory syndrome-related coronavirus
  source 1..222: note = BA.2.12.2-receptor binding domain
VQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYQYRLFRKSNLKPFERDISTEIYQAGNKPCNGVAGFNCYFPLRSYGFRPTYGVGHQPYRVVVLSFELLHAPATVCGPKKSTNLVKNKCVNF

SEQ ID NO: 8 - protein, 222 aa, Betacoronavirus Severe acute respiratory syndrome-related coronavirus
  source 1..222: note = BA.4-receptor binding domain
VQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNF

SEQ ID NO: 9 - protein, 222 aa, Betacoronavirus Severe acute respiratory syndrome-related coronavirus
  source 1..222: note = BA.5-receptor binding domain
VQPTESIVRFPNITNLCPFDEVFNATRFASVYAWNRKRISNCVADYSVLYNFAPFFAFKCYGVSPTKLNDLCFTNVYADSFVIRGNEVRQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNF

SEQ ID NO: 10 - protein, 419 aa, Betacoronavirus Severe acute respiratory syndrome-related coronavirus
  source 1..419: note = WT-N protein
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 11 - protein, 416 aa, Betacoronavirus Severe acute respiratory syndrome-related coronavirus
  source 1..416: note = B.1.1.529-N protein
MSDNGPQNQRNALRITFGGPSDSTGSNQNGGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSKRTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA